(19) United States
(12) Patent Application Publication
(10) Pub. No.: US 2021/0113733 A1
(43) Pub. Date: Apr. 22, 2021

(54) FIBROUS WOUND DRESSING COMPRISING AN ANTISEPTIC

(71) Applicant: Coloplast A/S, Humlebaek (DK)

(72) Inventors: Kent Hoeier Nielsen, Oelstykke (DK); Christian Aue Elbek, Virum (DK)

(21) Appl. No.: 17/254,311

(22) PCT Filed: Jul. 2, 2019
(86) PCT No.: PCT/DK2019/050214

§ 371 Date: Dec. 21, 2020

(30) Foreign Application Priority Data
Jul. 4, 2018 (DK) PA 2018 70459
Jan. 16, 2019 (DK) PA 2019 70033

(51) Int. Cl. A61L 15/48; A61L 15/22

(52) U.S. Cl. CPC A61L 15/48; A61L 15/225; A61L 2300/22; A61L 2300/216; A61L 2420/06; A61L 2300/404; A61L 2420/02; A61L 2300/232; A61L 2300/204

(57) ABSTRACT

A fibrous wound dressing comprising a formulation of amphiphilic antiseptic and separate surfactants is provided.

### TECHNICAL FIELD

The present technology relates to a fibrous wound dressing comprising an antiseptic formulation.

### BACKGROUND

Fibrous dressings for wound care are typically used for exuding wounds, including leg ulcers, pressure ulcers, diabetic foot ulcers, donor sites, postoperative wounds and skin abrasions.

A number of antiseptic compounds useful in wound treatment are amphiphilic, e.g. octenidine. Such compounds associate to surfaces, and have reduced mobility in a wound environment, or a hydrophilic matrix.

Additionally, challenges also exist when a formulation is exposed to a sensitive wound environment. In particular, the presence of ions and other components in the wound exudate can promote the undesirable precipitation of amphiphilic components.

As an amphiphilic molecule, octenidine has shown to associate to surfaces and thereby reduce mobility in a matrix. Early experiments documented that when octenidine is impregnated into a plain foam matrix, only a relatively low amount of octenidine was freely extractable (cf. experimental section). This strongly indicates that octenidine is attracted to the foam matrix, thereby restricting its release.

A need exists for a formulation of amphiphilic antiseptics, such as octenidine, in which the mobility of the amphiphilic antiseptic is increased in a wound environment. Additionally, the formulation should provide good solubility, mobility of the amphiphilic antiseptic and stability (i.e. lack of precipitation of the amphiphilic antiseptic). The present technology shows that the formulation of an amphiphilic antiseptic compound in a wound dressing can provide a major impact on the extractability, mobility and stability of said antiseptic.

### SUMMARY

A fibrous wound dressing is therefore provided which comprises a formulation of (a) an amphiphilic antiseptic and (b) at least one separate non-ionic surfactant or (c) at least one separate cationic surfactant or (d) at least one separate zwitterionic surfactant. The formulation can be coated on the surface of the fibrous wound dressing. The formulation may alternatively be comprised (i.e. impregnated) within the fibres of said fibrous wound dressing.

Additional aspects of the technology are presented in the following description, the examples and the dependent claims.

### DETAILED DISCLOSURE

As set out above, a fibrous wound dressing is provided comprising a formulation of (a) an amphiphilic antiseptic and (b) at least one separate non-ionic surfactant or (c) at least one separate cationic surfactant or (d) at least one separate zwitterionic surfactant. The term “separate” is used to mean that the same component may not be considered as both antiseptic and surfactant, but that the formulation comprises two separate, different components.

The amphiphilic antiseptic (component a) in the formulation—being amphiphilic—has both hydrophilic and hydrophobic moieties. Examples are quaternary ammonium compounds such as benzalkonium chloride and benzethonium chloride. Biguanides such as chlorhexidine or polyhexanide (PHMB) or other cationic compounds such as octenidine and ethyl lauroyl arginate (LAE). The antiseptic is preferably octenidine. The term “amphiphilic antiseptic” includes salts thereof.

Experimental results have shown that when octenidine is impregnated into a hydrophilic matrix, using hydrophilic polyurethane foam as model system only relatively low amount of octenidine was freely extractable (see Example 1, Table 1). This strongly indicates that octenidine is being attracted to the foam matrix, which thereby restricts the release of octenidine.

The limited release of octenidine from the foam matrix can possibly be explained on the basis of the chemical structure of octenidine. Octenidine consist of two pyridines and two aliphatic tails and an aliphatic linker between the pyridinium structure. This results in an abnormal structure for a cationic detergent (see FIG. 1) and a high degree of hydrophobicity. The high degree of hydrophobicity is expected to cause the attraction to surfaces and thereby low release. Similar reasoning can be applied to other amphiphilic antiseptics and to other substrates/products.

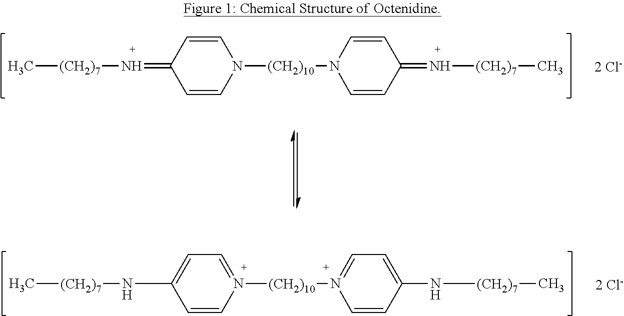

### Fibrous Wound Dressing

A fibrous wound dressing is provided. The term “fibrous” means comprised of fibres, typically in a nonwoven or woven structure, usually with physical entanglement between the fibres to maintain the integrity of the dressing.

In one alternative, the fibrous wound dressing comprises one or more layers of nonwoven material. Said layers of nonwoven material may be the same; or said layers may be different in terms of fibre type (natural, synthetic or semi-synthetic, or blends thereof), physical properties (e.g. hydrophilicity/hydrophobicity, physical dimensions or density) and/or type of nonwoven (e.g. airlaid, wetlaid, spunlace etc.). In one advantageous embodiment, the fibrous wound dressing comprises more than one layer of nonwoven material and/or more than one type of fibers.

Suitable fibres for the fibrous wound dressing include natural fibres such as wood, cotton, alginate collagen, or chitosan fibres, synthetic fibres such as polymeric fibres or semi-synthetic fibres such as rayon. Fibres may be staple fibres or continuous fibres.

Suitable nonwoven techniques for providing the layers of nonwoven material include airlaying, wet-laying and various spinning techniques.

The fibrous wound dressing may comprise additional components such as foams, super absorbent material or adhesive, typically in a layered construction. Alternatively, such components may be distributed throughout the fibrous dressing.

In another alternative, the fibrous wound dressing is a “stand alone” dressing in which the fibrous nonwoven layer is the sole component of the wound dressing.

The fibrous wound dressing has a wound-facing surface layer, which is defined as a sheet or layer arranged to be in direct contact to a wound, or peri-wound skin.

In embodiments, the fibrous wound dressing comprises carboxymethylcellulose (CMC) fibers and/or alginate-based fibers. Both CMC- and alginate-based fibrous dressings absorb water, thereby transforming from a distinct fiber structure to a more amorph gelling structure. Since this is an irreversible process that destroys the fiber structure, water cannot be used as a carrier for the impregnation solution for CMC- and alginate-based fibrous dressings. Therefore, the formulation for the fiber-based water absorbing platforms need to address this issue by not utilizing solvents that make the fibers transform and to choose a surfactant that following is dissolvable in the chosen solvent.

Ethanol or other polar organic solvents will not cause swelling alginate- and CMC-based fibers and is as such a good carrier solvent for the formulation since octenidine is readily dissolvable in ethanol. A range of relevant solvents are dissolvable in polar organic solvents Such as Tween 80. Tween 80 is a surfactant that has shown good performance together with Octenidine, both in relation to increasing release of octenidine, as well as stabilizing octenidine against precipitation with proteins and other wound bed compounds as well as being soluble in ethanol. Therefore, the octenidine formulation to be used for CMC and Alginate based fiber dressings is preferable composed from Ethanol as solvent, and Tween 80 as amphiphile surfactant. However, other surfactants dissolvable in alcohols, such as Tween 20, Empigen BB, benzalkonium chloride, or poloxamers, will potentially be candidates for this formulation system. In embodiments, the surfactant is Tween 80.

### Formulation

The fibrous wound dressing comprises a formulation of (a) an amphiphilic antiseptic and (b) at least one separate non-ionic surfactant or (c) at least one separate cationic surfactant. Preferably the surfactant is (b) at least one separate non-ionic surfactant or (d) at least one separate zwitterionic surfactant.

By formulation is meant a formulation solution that is meant to be impregnated into the fibrous wound dressing. Following impregnation, the carrying solvent is evaporated off, leaving the formulation compounds within the fiber structure. Thereby, the percental concentrations within the impregnation formulation can be re-calculated into mass of compound per square (or cubic) area of fiber, depending on the absorbance capacity of the given fiber. Example: If a fiber has a absorbency of 0.3 mL/square centimetre and the formulation holds 0.1% amphiphilic antiseptic and 1% non-ionic surfactant. Then, the fiber will be impregnated with 0.3 mg amphiphilic antiseptic and 3 mg non-ionic surfactant per square centimetre. This will lead to a finished fiber (dried) containing 0.3 mg amphiphilic antiseptic and 3 mg non-ionic surfactant per square centimetre. For reading this document, the relation between impregnation formulation and mass per square or cubic area fiber will be defined as in this section.

The antiseptic and the surfactant can either be blended into the matrix during formation of the fiber, applied during the formation of the fibrous sheet structure or applied as a coating or impregnation to the fibrous structure after formation of the fibrous structure.

The formulation is suitably a solution of said components in an appropriate solvent with good wettability to the applied fibrous dressing e.g. water and/or alcohols. Suitable alcohols may be methanol or ethanol or other polar organic solvents, when applying the said formulation to a hydrophilic fibrous dressing or mixtures with more apolar solvent such as hexane, ethyl acetate or volatile silicone fluids when applying said formulation to a hydrophobic fibrous dressing.

In one aspect, the formulation does not comprise surfactants other than the surfactants specified. In a further aspect, the formulation does not comprise antiseptics other than the antiseptic specified. In one aspect, the formulation consists of an amphiphilic antiseptic and at least one surfactant.

In one aspect, the formulation is free from inorganic salts. In particular, the formulation is free from halide salts of group I or II metals, e.g. NaCl, KCl, MgCl₂ or CaCl₂). Dissolution of the antiseptic is thereby improved. The formulation suitably comprises between 0.001-10% w/w, preferably between 0.05-5 wt % of said amphiphilic antiseptic. The formulation suitably comprises between 0.05-10% w/w, preferably between 0.01-5 wt %, more preferably between 0.1-5 wt % of said surfactant. The dressings and formulations can show antibacterial effects even at such low concentrations of antiseptic/surfactant. Meaning for a fiber dressing with an absorbency of 0.3 mL/cm²: 0.003-30 mg/cm² preferably between 0.15-3 mg/cm² of said amphiphilic antiseptic. The formulation suitably comprises between 0.15-30 mg/cm² w/w, preferably between 0.05-2.5 mg/cm², more preferably between 0.3-1.5 mg/cm² of said surfactant. By any deviation in exemplified absorbency (0.5 mL/cm²) the above-mentioned mass contents can be corrected.

In embodiments, the fibrous wound dressing comprises 0.003-30 mg/cm², preferably between 0.15-3 mg/cm², of said amphiphilic antiseptic. In embodiments, the fibrous wound dressing comprises between 0.15-30 mg/cm² w/w, preferably between 0.05-2.5 mg/cm², more preferably between 0.3-1.5 mg/cm² of said surfactant.

The formulation may be applied to a surface of the fibrous wound dressing which is arranged to face the user when in use (i.e. the opposite face to any backing layer). Alternatively, the formulation may be applied to a surface of the fibrous wound dressing which is arranged opposite the user when in use (i.e. the opposite face to the wound contact side). Alternatively or additionally, the formulation may be incorporated into the wound dressing (i.e. impregnated). Any known methods for applying the formulation into/onto the dressing may be used, such as rolling or spraying of the formulation onto a pre-formed fibrous wound dressing or incorporation by dipping/bathing the said fibrous wound dressing in the formulation.

In a first aspect, therefore a method for manufacturing a fibrous wound dressing is provided, said method comprising but not limited to
a. providing a formulation of (a) an amphiphilic antiseptic and (b) at least one separate non-ionic surfactant or (c) at least one separate cationic surfactant or (d) at least one separate zwitterionic surfactant, said formulation additionally including a solvent; and
b. applying the formulation to a pre-formed fibrous wound dressing, such that the formulation becomes coated on a surface of the fibrous wound dressing.

In another aspect, the formulation may be applied to free fibres, prior to formation of the fibrous wound dressing. A method for manufacturing a fibrous wound dressing is therefore provided, said method comprising
a. providing a formulation of (a) an amphiphilic antiseptic and (b) at least one separate non-ionic surfactant or (c) at least one separate cationic surfactant, or (d) at least one separate zwitterionic surfactant said formulation additionally including a solvent;
b. applying the formulation to fibers, such that the formulation becomes coated on said fibres; and
c. forming a fibrous wound dressing from said coated fibres.

As a further option, which may supplement the above options of coating the fibres/the wound dressing, the formulation may be comprised within the matrix of the fibres making up the wound dressing. In other words, the formulation (of antiseptic and surfactant) is blended with the fiber forming matrix, and then formed together with this material into the required fibres. In this manner, the formulation is encapsulated within the fibres of the fibrous wound dressing, which could provide improved properties with respect to stability and release of the antiseptic.

In this aspect, therefore, a method for manufacturing a fibrous wound dressing is provided, said method comprising
a. providing a formulation of (a) an amphiphilic antiseptic and (b) at least one separate non-ionic surfactant or (c) at least one separate cationic surfactant or (d) at least one separate zwitterionic surfactant, said formulation additionally including a solvent;
b. providing a polymer composition;
c. mixing said formulation with said polymer composition and spinning the mixture to form fibers impregnated with said formulation;
d. forming a fibrous wound dressing from said impregnated fibres.

The term “surfactant” as used herein means organic compounds that are amphiphilic, meaning they contain both hydrophobic groups and hydrophilic groups. The surfactant in the formulation is preferably non-ionic; i.e. it comprises polar hydrophilic regions which are not charged. It has been found that non-ionic surfactants can provide benefits in terms of stability of the formulation and release of the antiseptic.

Alternatively, the surfactant is cationic. It has been found that cationic surfactants can provide benefits in terms of stability of the formulation.

It has also been discovered that certain anionic detergents such as SDS, can interact with the antiseptic via ionic interaction and may cause precipitation and/or undesired interaction with the fibrous wound dressing.

In one aspect, the surfactant comprises a single hydrophobic moiety, and a single hydrophilic moiety. Without being bound by theory, it is hypothesised that surfactants having one of each of such moieties can arrange optimally with the amphiphilic antiseptic. Additionally, testing of certain surfactants with e.g. more than one hydrophobic moiety did not provide the desired benefits.

In one aspect, the surfactant is a fatty acid monoester or fatty acid monoamide of a polyhydroxy compound. If a monoamide surfactant is used, it should be uncharged in the physiological conditions present in a wound.

According to this aspect, the fatty acid monoester or fatty acid monoamide may comprise a C2-C22 fatty acid moiety, e.g. a C4-C18 fatty acid moiety or a C6-C12 fatty acid moiety. In embodiments, the fatty acid moiety is saturated. In embodiments, the fatty acid is unsaturated.

In another aspect, the surfactant is a fatty alcohol monoether of a polyhydroxy compound. The fatty alcohol monoether may comprise a C2-C22 fatty alcohol moiety, e.g. a C4-C18 fatty alcohol moiety or a C6-C12 fatty alcohol moiety. The fatty alcohol moiety may be saturated or unsaturated.

In embodiments, the fatty acid moiety or said fatty alcohol moiety used herein is saturated.

In embodiments, the fatty acid moiety or said fatty alcohol moiety used herein is unsaturated.

The polyhydroxy compound used as the hydrophilic moiety may be comprised of any multifunctional hydroxy- and/or amine compound (number of hydroxy groups+amine groups>=2), that may or may not be derivatized by any combination of ethylene oxide and propylene oxide. Particular polyhydroxy compounds may be selected from glycerol, sorbitan, ethoxylated sorbitan, glucose, ethylene glycol, polyethylene glycol or amine derivatives thereof.

Most preferably, the non-ionic surfactants are a C6-C12 fatty alcohol monoether of glucose, or a C6-C12 fatty acid monoester of ethoxylated sorbitan. Suitable non-ionic surfactants are e.g. polysorbates (Tween) and decyl glucoside.

In a further aspect, the surfactant is a di-block copolymer (A-B), wherein one block of said copolymer (A) is hydrophobic, and the other block (B) of said copolymer is hydrophilic.

In a further aspect, the surfactant is a block copolymer and preferable di-block copolymer (A-B), wherein one block of said copolymer (A) is hydrophobic, and the other block (B) of said copolymer is hydrophilic and preferably nonionic

The hydrophobic block (A) may be selected from, but not limited to, polypropylene oxide, polypropylene ethylenoxide copolymers, polysiloxanes, polystyrene, polylactide, polycaprolactone and the like. Similarly, the hydrophilic block may be selected from, but not limited to, polyethylene oxide, poly(ethylene oxide co-propylene oxide), polyoxazoline, poly(vinyl pyrolidone) and the like.

In embodiments, the surfactant is a zwitterionic surfactant, such as lauryl betaine (Empigen BB).

Overall, the surfactant may have a hydrophilic-lipophilic balance (HLB) between 10 and 17 inclusive.

### EXAMPLES

As an amphiphilic molecule, octenidine has shown to associate to surfaces and thereby reduce mobility in a matrix. Previous studies have indicated that Octenidine did not diffuse freely in foam matrices, indicating a high degree of interactions between octenidine and foam matrix.

To address this, we have investigated formulations to increase the mobility of octenidine by co-formulating different surface active compounds and salts. Solubility and stability (no precipitation when interacting with e.g. salts or proteins) was tested in a solution. Release from a fibrous wound dressing can be tested by coating or impregnating the formulation onto or into a fibrous matrix, optionally drying the fibrous and following carrying out release studies.

1. Octenidine in Foam, No Surfactant

Discs of hydrophilic polyurethane foam were impregnated by applying a known volume of octenidine-containing solution to the surface of the foam and letting it soak into the foam matrix in a liquid:foam ratio which allowed the foam to be saturated with liquid. Afterwards, the impregnated foam was dried at RT overnight.

The dried foam disc was immersed in the extraction media for 24 h and the extracted octenidine concentration was determined by UV at 285 nm

TABLE 1
These results show that when octenidine is impregnated into
a matrix such as a plain foam matrix only relatively
low amounts of octenidine were freely extractable.

| % Octenidine extractable after | | |
|---|---|---|
| being impregnated into plain | | Release % |
| foam w % OCT in | Release % | phosphate |
| impregnation solution | water | buffer (23 mM) |
| 0.1 w % | 5% | 7% |
| 0.5 w % | 1.5% | 13% |
| 1.0 w % | 1.5% | 20% |

2. Solubility of Octenidine with/without Surface Active Compounds.

In these experiments, octenidine dihydrochloride was dissolved in different solutions to determine the solubility with/without the presences of surface active compound (surfactant).

To investigate the interaction between the dissolved octenidine and isotonic salt concentrations (0.9%), 0.9% NaCl was co-formulated with glycerol (A4), tween (A5), or both combined (A6).

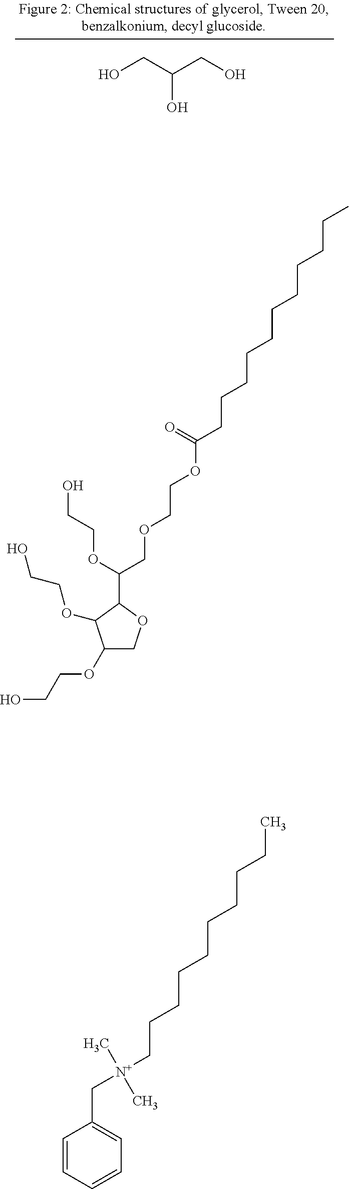 [SMILES: OCC(O)CO ; CCCCCCCCCCCC(=O)OCCOCC(OCCO)C1OCC(OCCO)C1OCCO ; CCCCCCCCCC[N+](C)(C)Cc1ccccc1]

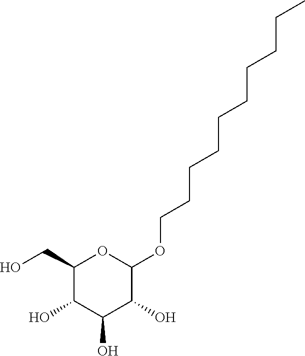 [SMILES: CCCCCCCCCCOC1O[C@H](CO)[C@@H](O)[C@H](O)[C@H]1O]

The solutions used were as follows:
A1: 3 w % Tween-20
A2: 5 w % glycerol
A3: 3 w % tween-20, 5 w % glycerol
A7. MQ water
A8: PBS buffer 23 mM
A9: 2% Benzalkonium chloride
A10: 5% Plantacare 2000 UP (50% decyl glucoside solution)

Two concentrations of Octenidine were tested: 1% and 3%
Conc. 1%: 1.00 g Octenidine+100 ml solution
Conc. 3%: 3.00 g Octenidine+100 ml solution

All solutions were prepared in conical glass bottles, sealed with plastic film at room temperature and stirred. The solutions were inspected every 15 min. and observations were recorded.

The results from the solubility tests are shown in Table 2:

TABLE 2
overview of solubilities of 1% Octenidine co-formulated
with different surfactant compounds.

| | | Total time to | | Dissolved after |
|---|---|---|---|---|
| 1% Octenidine | pH | dissolution | | 1 week at RT |
| Al | 3.23 | 1 | h | Yes |
| A2 | 5.06 | 1 h 05 min | | Yes |
| A3 | 3.28 | 1 h 15 min | | Yes |
| A7 | 4.99 | 1 h 20 min | | Yes |
| A8 | 6.82 | 41 | min | Yes |
| A9 | 6.45 | 1 h 15 min | | Yes |
| A10 | 10.14 | 51 | min | Yes |

All of the used solvent systems (H₂O, Glycerol, Phosphate, Tween20, Benzalkonium Cloride, and Plantacare (50% Decyl glucoside)) were able to dissolve 1% Octenidine. The solubility of 3% Octenidine was also tested and only Plantacare (solution A10) was able to fully dissolve 3% Octenidine and keep it in the dissolution without precipitation (results not shown).

Solvent systems containing salts (A4, A5, A6) did not dissolve 1% octenidine. Also, if octenidine is dissolved in respectively Tween20 or Tween20/glycerol, the same solubility/stability is indicated, while glycerol alone did not show any better solubilisation capacity than water alone. This indicates that glycerol does not have any significant effect on the solubility of octenidine, neither negative nor positive.

3. Stability of Solutions Towards Salts.

The solutions with 1% Octenidine from experiment 2 that were totally dissolved (A1, A2, A3, A7, A8, A9, A10), were tested in a new experiment. The solutions were diluted with 0.9% NaCl to different concentrations to observe whether the Octenidine precipitated in the solution. The ratios 2:1, 1:4 and 1:10 (test solution: 0.9% NaCl) were tested and all the solutions were heated to RT (37° C.) for 1 hour. To challenge the solubility, the samples were also cooled to 4° and possible precipitation was observed.

The results are shown in Table 3:

TABLE 3
Salt stability of octenidine solutions

| 1% | | Addition of | Addition of | Addition of |
|---|---|---|---|---|
| Octen- | | 0.9% NaCl | 0.9% NaCl | 0.9% NaCl |
| idine | Temp. | solution 2:1 | solution 1:4 | solution 2:1 |
| A1 | 37° C. | No precipitation | No precipitation | No precipitation |
| | 4° C. | Precipitation | Precipitation | Precipitation |
| A2 | 37° C. | Visible | Visible precipitation | — |
| | | precipitation | | |
| | 4° C. | — | — | — |
| A3 | 37° C. | No precipitation | No precipitation | No precipitation |
| | 4° C. | Precipitation | Precipitation | Precipitation |
| A7 | 37° C. | Visible | Visible precipitation | — |
| | | precipitation | | |
| | 4° C. | — | — | — |
| A8 | 37° C. | No precipitation | Visible precipitation | — |
| | 4° C. | Precipitation | — | — |
| A9 | 37° C. | No precipitation | No precipitation | No precipitation |
| | 4° C. | Precipitation | Precipitation | Precipitation |
| A10 | 37° C. | No precipitation | No precipitation | No precipitation |
| | 4° C. | — | Less precipitation | Precipitation |
| | | | than other | |
| | | | solutions | |

If addition of salt is carried out after octenidine has been dissolved, the precipitating effect of NaCl is not seen at room temperature for solutions A1, A3, A9 and A10 (Table 3), indicating that an interaction between an amphiphile such as Tween20 or decyl glucoside and octenidine, protects octenidine from salt precipitation.

For all formulations except Plantacare precipitations was observed at octenidine: salt solution of 2:1 at increasing salt concentrations (1:4) slight precipitation was observed in the octentine:plantacare formulation and with even stronger precipitations at a ratio of 1:10. However, this show that decylglucoside has the best capacity to stabilize octenidine in relation to salting out.

Overall, the 3 amphiphiles (Tween 20, benzalkonium and decyl glucoside) all dissolve 1% octenidine. But most importantly, indicated by the salt additions, they are able to stabilize octenidine in a salt-containing solution such as a wound bed and avoid precipitation upon contact with salt. Based on the temperature experiments it is indicated that decyl glucoside (Plantacare) has the best capacity to stabilize the octenidine.

4. Protein Binding and Precipitation

The purpose of this experiment is to investigate the capability of surfactant to protect Octenidine from precipitation when mixed with a protein/salt media, such as simulated wound fluid (SWF), to further understand how Octenidine and the co-formulation with detergents will respond to being released into a wound bed environment.

The results show that surfactants can significantly reduce the interaction between a protein pool and Octenidine by reducing the agglomeration of octenidine and proteins/salts. This means that the surfactants will prevent unwanted precipitation, thereby making sure that a large portion of the Octenidine is available for acting in the wound environment.

The following surfactants were tested:

| Solution | | | |
|---|---|---|---|
| no. | Surfactant: | INCI name | Batch no. |
| A | 1% Tween 20 | Polysorbate 20 | Batch #094K0052 |
| B | 1% Tween 80 | Polysorbate 80 | Lot #BCBV7863 |
| C | 1% Plantacare 810 UP | Caprylyl/Capryl | lot. 17483268 |
| | | Glucoside | |
| D | 1% Plantacare 2000 UP | Decyl Glucoside | lot. 0019096298 |
| E | 1% Benzalkonium | Benzalkonium | Lot #BCBV7858 |
| | chloride | chloride | |
| F | 1% Empigen BB | Lauryl Betaine | Lot #BCBQ6967 |
| G | 1% Decanesulfonate | Decane- | Lot #BCBT6967 |
| | | sulfonate | |
| H | 1% Plantacare 1200 UP | Lauryl glucoside | lot. 19090815 |
| I | Water | — | — |

The experiment was done as follows:
i) 2 ml of solution A, B, C etc., each containing 1 mg/ml Octenidine, were mixed with 2 ml SWF or water. The mix of solutions were done twice (one for each filter type).
ii) The mix of solutions were incubated for 1 hour at room temp. on a shaking table at 100 rpm.
iii) The mix of solutions were filtrated through a 0.22 μm filter.
iv) The filtrated solution was diluted ten times in eluent. The Octenidine conc. should be 0.05 mg/ml (to be within detection area) if 100% was recovered after incubation and filtration.
v) Controls were prepared by diluting the formulation solution in eluent (50% McIlvaine buffer/50% Methanol) to conc. 0.05 mg/ml (dilution ×20).
vi) The samples and controls were analysed using HPLC.

The results were as follows.

| Solution | | | Recovery in | Recovery in |
|---|---|---|---|---|
| no. | Surfactant: | Octenidine | SWF (%) | water (%) |
| A | 1% Tween 20 | 1 mg/ml | 99 | 100 |
| B | 1% Tween 80 | 1 mg/ml | 100 | 100 |
| C | 1% Plantacare 810 UP | 1 mg/ml | 71 | 100 |
| D | 1% Plantacare 2000 | 1 mg/ml | 69 | 100 |
| | UP | | | |
| E | 1% Benzalkonium | 1 mg/ml | 47 | 100 |
| | chloride | | | |
| F | 1% Empigen BB | 1 mg/ml | 97 | 100 |
| G | 1% Decanesulfonate | 1 mg/ml | 7 | n.a. |
| H | 1% Plantacare 1200 | 1 mg/ml | 54 | 99 |
| | UP | | | |
| I | Water | 1 mg/ml | 26 | 100 |

The results show that Octenidine is precipitated by mixing with protein and salt containing solutions as well as when formulated with anionic surfactants, such as decanosulfonate. However, when co-formulated with nonionic (plantacare, Tween), cationic (Benzalkonium chloride) or zwitterionic (Empigen) surfactants, Octenidine is protected against precipitation, most probably by hydrophobic-hydrophobic interaction between octenidine and detergents, scavenging the octenidine molecule from interacting with salts and/or proteins.

5. Release of Octenidine from Fibrous Wound Dressings

This example was carried out to gain an understanding of how to introduce an amphiphilic antiseptic, such as octenidine, into a fiber-based wound dressing based, e.g., on alginates or hydrofibers, such as carboxymethylcellulose (CMC). As a hydrophobic molecule, octenidine has shown to associate to surfaces and thereby reduce mobility in a matrix. To overcome this, it was investigate whether formulations with increased mobility of octenidine could be achieved by co-formulating with a surfactant.

The experiment was conducted Coloplast Biatain alginate wound dressings and a generic CMC based wound dressing and on free fibers of CMC. Fibers and dressings were prepared according to the below table.

| Sample | Substrate | Octenidine | Tween 80 |
|---|---|---|---|
| 1 | CMC dressing | 2 mg/ml | 0% |
| 2 | CMC dressing | 2 mg/ml | 2% |
| 3 | Alginate dressing | 2 mg/ml | 0% |
| 4 | Alginate dressing | 2 mg/ml | 2% |
| 5 | CMC fiber | 0.1% | 0% |
| 6 | CMC fiber | 0.1% | 2% |

For the dressings, a sample is punched out with Ø20 mm, which sample is then impregnated with solutions of 2 mg/ml OCT in ethanol, 2 mg/ml OCT in ethanol with 2% Tween 80, or 2% Tween 80 in ethanol (without OCT). The samples were placed in Petri dishes and added 2×500 microliters impregnation solution for the CMC and 1×500 microliters for alginate. The samples were left to dry in the fume hood over the night.

For the fibers, 30 g of fiber was dipped into a solution containing either 0.1% octenidine (OCT) in EtOH or 0.1% OCT in a solution of 2% Tween 80 in EtOH. The samples were dipped for 5 minutes and thereafter dried.

The extraction was carried out as follows:
For the dressing samples: The impregnated disk of the dressings is placed in 50 ml centrifuge tubes.
0.5 g of fibers where weighed off and put into a polypropylene tea bag which was following heat sealed. The fiber containing teabag was following placed in a 50 mL centrifuge tube.
Each tube is added 10 ml of PBS buffer pH 7,4
The tubes are placed at a shaking table at 100 rpm and after 24 hours the pieces of fiber sample are carefully transferred to a new 50 ml centrifuge tube containing 10 ml PBS.
After 24, 48 and 72 hours the same procedure is repeated, but after 72 hours the samples are instead thrown out.
The test tubes containing the release media at time point 24, 48 and 72 hours are analysed by UV measurement for determination of the concentration of Octenidine. Samples where filtered at a 0.45 μm to filter out any solid material that could interfere with UV measurement.
For Alginate dressings, measurements were only carried out for 48 h, due to starting disintegration of the sample.

The results are as shown in the table below. Recovery is given as total accumulated recovery, i.e. the sum of the recovery at the indicated time point and previous time point(s), in percentage of the total impregnated octenidine.

| | | | | Recov- | Recov- | Recov- |
|---|---|---|---|---|---|---|
| | | | Tween- | ery | ery | ery |
| Sample | Description | OCT | 80 | (24 h) | (48 h) | (72 h) |
| 1 | CMC | + | − | 8% | 13% | 18% |
| | dressing | | | | | |
| 2 | CMC | + | + | 82% | 88% | 90% |
| | dressing | | | | | |
| 3 | Alginate | + | − | 12% | 18% | — |
| | dressing | | | | | |
| 4 | Alginate | + | + | 86% | 91% | — |
| | dressing | | | | | |
| 5 | CMC fiber | + | − | 1% | 4% | 6% |
| 6 | CMC fiber | + | + | 88% | 100% | 100% |

The results show that octenidine can be co-formulated with a surfactant (Tween 80) and that this co-formulation increases its mobility in fiber-based wound dressings. Ethanol was used as a solvent and fibers could be wetted and dried without visible changes. The surfactant (Tween 80) was dissolved into ethanol, together with octenidine, and significantly increases the release of octenidine from the impregnated dressings and fibers.

### CONCLUSIONS

Formulating octenidine with non-ionic or cationic or zwitterionic surfactants—preferably non-ionic surfactants—increases the mobility and stability of the octenidine. Formulating with Decyl glucoside (plantacare) resulted in the highest amount of total release octenidine with a total amount of released octenidine reaching 85% at 72 h together with an increased stability to salts. The results show that amphiphilic compounds can interact with octenidine and increase stability of octenidine. Highest mobility and stability increase was seen when using decyl glucoside (Plantacare) followed by Tween 20. Glycerol did not have any effect on octenidine mobility or stability, while NaCl caused precipitation, if octenidine had not been stabilized by amphiphiles before adding salts.

The experiments show that the nature of the surfactant must be carefully considered when providing formulations for fibrous wound dressings.

Although the invention has been illustrated with reference to a number of embodiments, aspects and examples, the skilled person can combine such embodiments, aspects and examples within the scope of the appended claims.

1.-24. (canceled)

25. A wound dressing comprising a formulation of (a) an amphiphilic antiseptic and (b) a non-ionic surfactant or (c) a cationic surfactant or (d) a zwitterionic surfactant; and
a fibrous dressing.

26. The wound dressing according to claim 25, wherein the non-ionic surfactant comprises a fatty acid monoester or fatty acid monoamide of a polyhydroxy compound.

27. The wound dressing according to claim 26, wherein the fatty acid monoester or fatty acid monoamide comprises a C2-C22 fatty acid moiety, a C4-C18 fatty acid moiety or a C6-C12 fatty acid moiety.

28. The wound dressing according to claim 27, wherein the polyhydroxy compound comprises glycerol, sorbitan, ethoxylated sorbitan, glucose, ethylene glycol, polyethylene glycol or amine derivatives thereof.

29. The wound dressing according to claim 25, wherein the non-ionic surfactant comprises a fatty alcohol monoether of a polyhydroxy compound.

30. The wound dressing according to claim 28, wherein the fatty alcohol monoether comprises a C2-C22 fatty alcohol moiety, a C4-C18 fatty alcohol moiety or a C6-C12 fatty alcohol moiety.

31. The wound dressing according to claim 30, wherein the polyhydroxy comprises glycerol, sorbitan, ethoxylated sorbitan, glucose, ethylene glycol, polyethylene glycol or amine derivatives thereof.

32. The wound dressing according to claim 25, wherein the non-ionic surfactant comprises a di-block copolymer (A-B), wherein one block of the copolymer (A) is hydrophobic, and the other block (B) of the copolymer is hydrophilic.

33. The wound dressing according to claim 32, wherein the hydrophobic block (A) comprises a polypropylene oxide, a polypropylene ethylene oxide copolymer, a polysiloxane, a polystyrene, a polylactide, or a polycaprolactone.

34. The wound dressing according to claim 32, wherein the hydrophilic block (B) comprises a polyethylene oxide, a poly(ethylene oxide co-propylene oxide), a polyoxazoline or a poly(vinyl pyrrolidone).

35. The wound dressing according to claim 25, wherein the formulation comprises a solution of the components in water and/or alcohols.

36. The wound dressing according to claim 25, wherein the amphiphilic antiseptic comprises benzalkonium chloride, benzethonium chloride, chlorhexidine, polyhexanide (PHMB), octenidine or ethyl lauroyl arginate (LAE) or salts thereof.

37. The wound dressing according to claim 25, wherein the amphiphilic antiseptic comprises octenidine or salts thereof.

38. The wound dressing according to claim 25, wherein the formulation is coated on a surface of the fibrous dressing or within fibres of the fibrous dressing.

39. The wound dressing according to claim 25, wherein the non-ionic surfactant is decyl glucoside and the amphiphilic antiseptic is octenidine or salts thereof.

40. The wound dressing according to claim 38, wherein the fibrous dressing is a carboxymethylcellulose or an alginate.

41. The wound dressing according to claim 25, wherein the formulation comprises between 0.001 and 10% w/w of the amphiphilic antiseptic.

42. The wound dressing according to claim 41, wherein the formulation comprises between 0.05 and 10% w/w of the surfactant.

43. A method for manufacturing a wound dressing comprising the steps:
a. providing a formulation comprising (a) an amphiphilic antiseptic and (b) a non-ionic surfactant or (c) a cationic surfactant, or (d) a zwitterionic surfactant wherein the formulation further comprises a solvent; and
b. applying the formulation to a pre-formed fibrous wound dressing, such that the formulation becomes coated on a surface of the fibrous wound dressing; and
c. optionally, forming a fibrous wound dressing from the coated fibres.

44. A method for manufacturing a wound dressing comprising the steps:
a. providing a formulation comprising (a) an amphiphilic antiseptic and (b) a non-ionic surfactant or (c) a cationic surfactant, or (d) a zwitterionic surfactant wherein the formulation further comprises a solvent;
b. providing a polymer composition;
c. mixing the formulation with the polymer composition and spinning the mixture to form fibers impregnated with the formulation; and
d. forming a fibrous wound dressing from the impregnated fibres.
